(19) United States
(12) Patent Application Publication
(10) Pub. No.: US 2003/0168192 A1
(43) Pub. Date: Sep. 11, 2003

(54) Novel monomers, polymers thereof and the use of the polymers

(76) Inventor: Amjad Mohmood Mohammed, Bradford (GB)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/343,676

(22) PCT Filed: Jul. 27, 2001
(86) PCT No.: PCT/EP01/08703

(30) Foreign Application Priority Data
Aug. 9, 2000 (GB) 0019415.9

(51) Int. Cl. D21H 17/33; D21H 17/45; C07D 265/30; C07D 215/12

(52) U.S. Cl. 162/158; 162/168.2; 162/164.1; 526/258; 526/260; 544/224; 544/264; 544/358; 544/336; 544/59; 544/171; 544/332; 546/335; 546/176; 548/350.1; 548/494; 548/462; 548/572

(57) ABSTRACT

Compounds of formula (1) containing sterically hindered groups, polymers thereof and the use of these polymers in papermaking processes and dewatering processes.

The present invention relates to monomers which are relatively sterically hindered, polymers thereof and the use of such polymers.

In the papermaking art, an aqueous suspension containing cellulosic fibres, and optional fillers and additives, referred to as stock, is fed into a headbox which ejects the stock onto a forming wire. Water is drained from the stock through the forming wire so that a wet web of paper is formed on the wire, and the web is further dewatered and dried in the drying section of the paper machine. Water obtained by dewatering the stock, referred to as white water, which usually contains fine particles, e.g. fine fibres, fillers and additives, is normally recirculated in the papermaking process. Drainage and retention aids are conventionally introduced into the stock in order to facilitate drainage and increase adsorption of fine particles onto the cellulosic fibres so that they are retained with the fibres on the wire. Cationic organic polymers like cationic starch and cationic acrylamide-based polymers are widely used as drainage and retention aids. Such polymers may also be used as dewatering aids in sewage sludge treatment processes.

These polymers can be used alone but more frequently they are used in combination with other polymers and/or with anionic microparticulate materials such as, for example, anionic inorganic particles like colloidal silica, colloidal aluminium-modified silica and bentonite.

U.S. Pat. Nos. 4,980,025; 5,368,833; 5,603,805; and 5,607,552; European Patent Application Number 752,496; and International Patent Application Publication Number WO 97/18351 disclose the use of cationic and amphoteric acrylamide-based polymers and anionic inorganic particles as stock additives in papermaking. Similar systems are disclosed in European Patent Application Number 805,234. International Patent Application Publication Number WO 99/55965 discloses the use of a cationic polymer having an aromatic group.

It has, for example in International Patent Application Publication Number WO 99/55965, been observed that the performance of drainage and retention aids comprising cationic organic polymers is deteriorated when used in stocks with high levels of salts, i.e. high conductivity, and dissolved and colloidal substances. Higher dosages of cationic polymer are normally required in such stocks but usually the drainage and retention effect obtained is still not entirely satisfactory. These problems are even more pronounced in paper mills where white water is extensively recirculated with the introduction of only low amounts of fresh water into the process, thereby further increasing the accumulation of salts and colloidal materials in the white water and the stock to be dewatered.

Surprisingly, it has been found that the introduction of a sterically hindered group into these types of polymer prevent the polymer chain from collapsing on itself, i.e. keeping the chain as extended as possible, in electrolyte environments, and show superior results over known polymers when evaluated as a retention and a drainage aid.

According to the present invention it has been found that improved drainage and retention can be obtained in stocks containing high levels of salt (high conductivity) and colloidal materials when using drainage and retention aids comprising a cationic organic polymer produced from a relatively sterically hindered monomer.

The first aspect of this invention relates to the previously mentioned monomer, which is a compound of the formula (1):

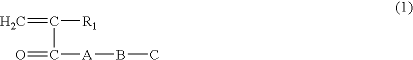

wherein R₁ is H or CH₃, A is O or NH, B is an alkylene group of from 0 to 10 carbon atoms or a hydroxy alkylene group, and C is a cyclic group bonded to A when B is 0, or B by a nitrogen atom which is in the quaternised form.

A is preferably an oxygen atom. B is preferably an alkylene group of from 2 to 4 carbon atoms.

C is preferably a relatively bulky, or sterically hindered group. Preferably, C is a cyclic group of the formula (2):

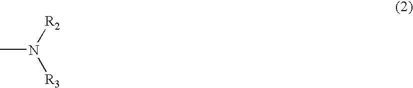

wherein R₂ and R₃ form, together with the adjacent nitrogen atom, a cyclic group which may be saturated or unsaturated, and may contain hetero atoms within the cyclic group or as substituents on the cyclic group, and may also contain lower alkyl groups.

For example, C may be selected from the group consisting of pyrrolidine, pyrrolidine N-substituted by C₁ to C₄ alkyl, pyrrolidinyl, pyrroline, pyrrolinyl, imidazolidine, imidazolidinyl, imidazoline, imidazolinyl, pirazolidine, pirazolidinyl, pirazoline, pirazolinyl, piperidine, piperidyl, piperazine, piperazine N-substituted by C₁ to C₄ alkyl, piperazinyl, indoline, indolinyl, isoindoline, isoindolinyl, quinuclidine, quinuclidinyl, morpholine, morpholinyl, 2H-pyrrole, 2H-pyrrolyl, pyrrole, pyrrolyl, imidazole, imidazolyl, pyrazole, pyrazolyl, pyridine, pyridyl, pyrazine, pyrazinyl, pyrazine, pyrazine para-substituted by C₁ to C₄ alkyl, pyrazinyl, pyrimidine, pyrimidinyl, pyradizine, pyridaznyl, indolizine, indolizinyl, isoindole, isoindolyl, 3H-indole, 3H-indolyl, indole, indolyl,1H-indazole, indazolyl, purine, purinyl, 4H-quinolizine, 4H-quinolizinyl, isoquinoline, isoquinolyl, quinoline, quinolyl, phthalazine, phthalazinyl, naphthyridine, naphthyridinyl, quinoxaline, quinoxalinyl, quinazoline, quinazolinyl, cinnoline, cinnolinyl, pteridine, pteridinyl, 4aH-carbazole, 4aH-carbazolyl, carbazole, carbazolyl, carboline, carbolinyl, phenanthridine, phenanthridinyl, acridine, acridinyl, perimidine, perimidinyl, phenanthroline, phenanthrolinyl, phenazine, phenazinyl, phenarsazine, phenarsazinyl, phenothiazine, henothiazinyl, furazan, furazanyl, phenoxazine, phenoxazinyl, isothiazole, isoxazole, proline or dehydroproline.

R₂ and R₃ are preferably C₁ to C₃ alkyl groups, more preferably C₂ to C₃ alkyl groups which are bonded together by a heteroatom. More preferably C is a morpholine group.

Compounds used to quatemise the nitrogen of group C which is bonded to group B, may be selected from any of the known counterions or alkylating agents. The counterion may be selected from the group consisting of alkyl halides, aryl halides, aralkyl halides, cyclo-alkyl halides, alkyl sulphate, dialkyl sulphate and other known counterions such as ammonium halides.

Preferably, the counterion used to quaternise the monomer is selected so that the resulting cationic charge remains after any changes in pH. For example, the use of hydrogen chloride to quaternise the monomer would result in a cafionic compound which would not retain its' charge after a change in pH, i.e. the cationic monomer would revert back to the nonionic form. Preferred counterions include alkyl halides and aralkyl halides, more preferably methyl chloride and benzyl chloride.

A second aspect of the invention relates to methods of preparing a compound of formula (1) wherein A is an oxygen atom. One method of preparing a compound of formula (1),

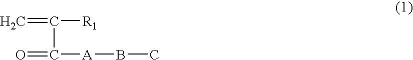

R₁ is H or CH₃, A is O, B is an alkylene group of from 0 to 10 carbon atoms or a hydroxy alkylene group, and C is a cyclic group bonded to A when B is 0, or B by a nitrogen atom which is in the quaternised form, comprises reacting an acid of the formula (3),

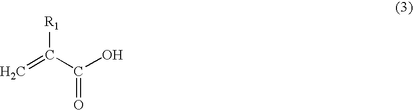

with an alcohol of the formula (4),

HO—B—C (4)

in the presence of an acid catalyst and under reflux conditions. This reaction may be brought to completion by removal of the water formed in the preparation. Choices for the catalyst include sulphuric acid, hydrogen chloride, p-toluenesulphonic acid, orthophosphoric acid, dibutyl tin oxide and other known acidic catalysts.

Another method of preparing a compound of formula (1),

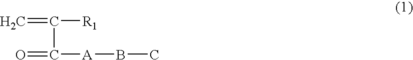

R₁ is H or CH₃, A is O, B is an alkylene group of from 0 to 10 carbon atoms or a hydroxy alkylene group, and C is a cyclic group bonded to A when B is 0, or B by a nitrogen atom which is in the quaternised form, comprises reacting an acid chloride of the formula (5),

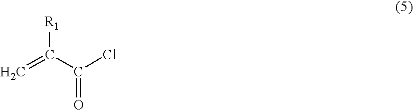

with an alcohol of the formula (4).

HO—B—C (4)

This reaction produces hydrogen chloride as a by product which will need to be trapped by a base, such as a tertiary amine.

Another method of preparing a compound of formula (1),

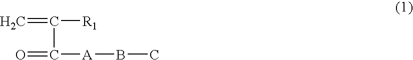

R₁ is H or CH₃, A is O, B is an alkylene group of from 0 to 10 carbon atoms or a hydroxy alkylene group, and C is a cyclic group bonded to A when B is 0, or B by a nitrogen atom which is in the quatemised form, comprises reacting an acid of the formula (3),

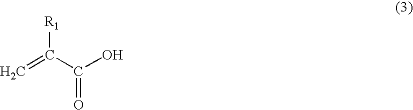

with a halide of the formula (6).

X—B—C (6)

Another method of preparing a compound of formula (1),

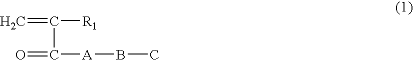

R₁ is H or CH₃, A is O, B is an alkylene group of from 0 to 10 carbon atoms or a hydroxy alkylene group, and C is a cyclic group bonded to A when B is 0, or B by a nitrogen atom which is in the quatemised form, comprises reacting an acid of the formula (3),

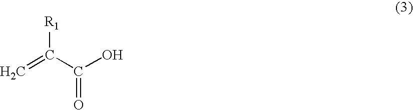

with an olefin of the formula (7),

H₂C═B—C (7)

in the presence of an acid catalyst.

Another method of preparing a compound of formula (1),

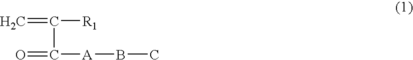

R₁ is H or CH₃, A is O, B is an alkylene group of from 0 to 10 carbon atoms or a hydroxy alkylene group, and C is a cyclic group bonded to A when B is 0, or B by a nitrogen atom which is in the quaternised form, which comprises reacting a nitrile of the formula (3),

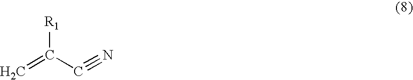

with an alcohol of the formula (4),

HO—B—C (4)

in the presence of an acid catalyst.

A preferred method of preparing a compound of formula (1),

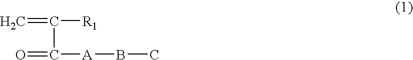

R₁ is H or CH₃, A is O or NH, B is an alkylene group of from 0 to 10 carbon atoms or a hydroxy alkylene group, and C is a cyclic group bonded to A when B is 0, or B by a nitrogen atom which is in the quaternised form, which comprises reacting an ester of the formula (9),

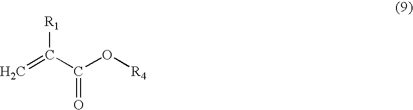

wherein R₄ is a C₁ to C₄ alkyl, with an alcohol of the formula (4).

HO—B—C (4)

This method is of particular interest as it offers a “one pot” reaction, with no isolation and purification of intermediates needed. The reagents should be dried by azeotropic distillation, then refluxed in the presence of a catalyst such as titanium tetraisoperoxide.

A further aspect of the invention relates to methods of preparing a compound of formula (1) wherein A is NH. One method of preparing a compound of formula (1),

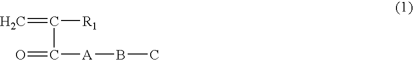

R₁ is H or CH3, A is NH, B is an alkylene group of from 0 to 10 carbon atoms or a hydroxy alkylene group, and C is a cyclic group bonded to A when B is 0, or B by a nitrogen atom which is in the quaternised form, comprises reacting an acid chloride of the formula (5),

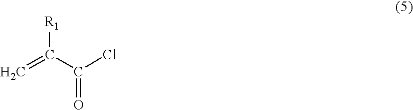

with an amine of the formula (10).

NH—B—C (10)

Another method of preparing a compound of formula (1),

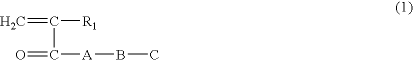

R₁ is H or CH₃, A is NH, B is an alkylene group of from 0 to 10 carbon atoms or a hydroxy alkylene group, and C is a cyclic group bonded to A when B is 0, or B by a nitrogen atom which is in the quaternised form, comprises reacting an acid chloride of the formula (11),

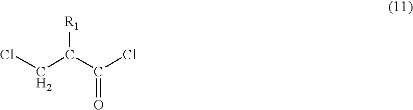

with an amine of the formula (10).

NH—B—C (10)

The intermediate thus formed is then treated with a base, such as sodium hydroxide, to give the final product.

Where the previously mentioned syntheses produce a compound of formula (1) in which the nitrogen atom of group C which is bonded to group A when B is 0, or B which is in a quaternised form the uncharged monomer is quaternised with a known counterion, using a suitable solvent, such as acetone.

Other known methods for preparing these monomers may be used.

The counterion may be selected from the group consisting of alkyl halides, aryl halides, aralkyl halides, cyclo-alkyl halides, alkyl sulphate, dialkyl sulphate and other known counterions such as ammonium halides. Preferred counterions include alkyl halides and aralkyl halides, more preferably methyl chloride and benzyl chloride.

A further aspect of the invention relates to a cationic or amphoteric organic polymer comprising in polymerised form a monomer containing group C.

The group C of the polymer may be present in the polymer backbone or, preferably, it can be a pendant group attached to or extending from the polymer backbone or be present in a pendant group that is attached to or extending from the polymer backbone.

Preferably, the polymer is a cationic or amphoteric organic polymer comprising in polymerised form a monomer of the formula (1), as described previously.

The polymer may have a specific viscosity of from 1 to 20 dl/g, preferably from 4 to 14 dl/g, more preferably from 5 to 10 dl/g. Specific viscosities mentioned in this patent are measured at a pH of 7 and at an active polymer concentration of 0.02%

The polymer may be a homopolymer or may additionally contain other copolymerizable materials.

The polymer is preferably prepared from a monomer mixture comprising from 10 to 100 mole % of a monomer of formula (1) which is in a cationic form as previously described, and from 0 to 90 mole % of other copolymerizable materials.

The polymer is more preferably prepared from a monomer mixture comprising from 30 to 80 mole % of a monomer of formula (1) which is in a cationic form as previously described and from 70 to 20 mole % of other copolymerizable materials.

For use in the area of paper manufacture, the polymer is more preferably prepared from a monomer mixture comprising from 20 to 40 mole % of a monomer of formula (1) which is in a cationic form as previously described and from 80 to 60 mole % of other copolymerizable materials.

For use in the sewage sludge dewatering area of industry, the polymer is more preferably prepared from a monomer mixture comprising from 50 to 100 mole % of a monomer of formula (1) which is in a cationic form as previously described and from 50 to 0 mole % of other copolymerizable materials.

Such copolymerisable materials may include at least one ethylenically unsaturated monomer.

One or more ethylenically unsaturated monomers may be selected from the group consisting of (meth)acrylamide, N-alkyl (meth)acrylamides and N,N-dialkyl (meth)acrylamides, dialkylaminoalkyl (meth)acrylamides, dialkylaminoalkyl (meth)acrylates, vinylamides, acid addition salts and quaternary ammonium salts of the dialkylaminoalkyl (meth)acrylates, acrylic acid, methacrylic acid, diallyl dialkylammonium chloride and other salts thereof, and sulfonated vinyl addition monomers. Quaternaries and salts of such monomers may also be used.

The preferred number of ethylenically unsaturated monomer units comprising the polymer is from one to three.

Preferred comomoners include (meth)acrylamide and dialkylaminoalkyl(meth)acrylates. More preferred comonomers include acrylamide and dimethylaminoethyl(meth)acrylate quaternary ammonium salts.

Amphoteric polymers may be produced from a monomer of a formula (1) and combinations of ethylenically unsaturated cationic and anionic monomers, and optional non-ionic monomers. Examples of known anionic monomers include sodium acrylate and sodium methacrylate.

A polymer of particular interest is prepared from a monomer mixture comprising from 20 to 40 mole % of a monomer of formula (1)

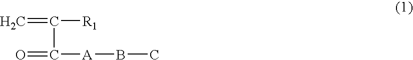

wherein R₁ is H or CH₃, A is O, B is an ethylene group, and C is a morpholine group bonded to B by a nitrogen atom, which is in the quaternised form, and from 80 to 60 mole % of acrylamide.

The charge density of the polymer may be from 1.0 to 4.0 meq/g of dry polymer, and is preferably from 1.5 to 3.0 meq/g.

A charge density of from 1.5 to 1.7 meq/g may be useful for polymers used in paper manufacture.

A charge density of from 2.0 to 5.0 meq/g may be useful for polymers used in sewage sludge dewatering.

The polymer may be in a solid form, such as a powder or bead. The polymer may also be in a liquid form, such as a solution, emulsion or dispersion. The polymer may also be in a gel form.

The polymer may be made by any known suitable polymerisation process, although a reverse phase bead polymerisation process is preferred. The polymer may be linear, branched or crosslinked.

A branching agent makes it possible to impart a branched structure to the acrylamide-based polymer, e.g. by copolymerisation of a monomer mixture including a monomeric branching agent containing ethylenically unsaturated bond(s) and/or by reaction between other types of reactive group(s) present in a branching agent with reactive group(s) present in the acrylamide-based polymer during or after polymerisation. Examples of suitable branching agents include compounds having at least two, and preferably two, ethylenically unsaturated bonds; compounds having at least one ethylenically unsaturated bond and at least one reactive group; and compounds having at least two reactive groups. Examples of suitable reactive groups include epoxides, aldehydes, and hydroxyl groups. It is preferred that the branching agent is difunctional i.e., that there are two groups of the type ethylenically unsaturated bond and/or reactive group present in the branching agent. Preferably the acrylamide based polymer contains, in polymerised form, at least one ethylenically unsaturated monomer functioning as a branching agent, and more preferably the branching agent has two ethylenically unsaturated bonds.

Examples of suitable monomeric branching agents containing two ethylenically unsaturated bonds include alkylene bis(meth)acrylamides, e.g. methylene bisacrylamide and methylene bismethacrylamide, diacrylates and dimethacrylates of mono-, di- and polyethylene glycols, allyl- and vinyl-functional (meth)acrylates and (meth)acrylamides, e.g.: N-methyl allylacrylamide and N-vinyl acrylamide, and divinyl compounds, e.g. divinyl benzene. Examples of suitable monomeric branching agents containing one ethylenidally unsaturated bond and one reactive group include glycidyl acrylate, methylol acrylamide and acrolein. Examples of branching agents containing two reactive groups include glyoxal, diepoxy compounds and epichlorohydrin.

The polymer may be crosslinked. Covalent or ionic cross linking agents may be used. Suitable covalent cross linking agents are polyethylenically unsaturated monomers such as methylene bis acrylamide, the di-, tri- or polyacrylates (e.g., diethylene glycol diacrylate, trimethyol propane triacrylate, and polyethylene glycol diacrylate where the polyethylene glycol typically has a molecular weight of 200, 400 or 600) and ethylene glycol diglycidyl ether, or any of the other polyethylenically unsaturated monomers conventionally used for cross linking polymers formed from ethylenically unsaturated water soluble monomers.

It is sometimes preferred to conduct the polymerisation in the presence of ionic cross linking agent. This may cross link with acrylamide or anionic groups in the monomer or with anionic groups in the reagent or both. Suitable ionic cross linking agents that may be used include aluminium or zirconium salts or other tri or higher polyvalent metal ions.

The amount of such cross linking agents, based on the dry weight of monomer, is generally in the range from 0.01 to 1,000 parts per million (ppm), preferably from 0.01 to 500 ppm, more preferably from 0.1 to 60 ppm.

The polymers of the present invention may be used in a papermaking process as a retention aid or drainage aid, in a sewage sludge treatment process as a dewatering aid or as a rheology modifier.

A further aspect of the invention relates to a process for the production of paper from a suspension containing cellulosic fibres, and optional fillers, which comprises adding to the suspension a cationic organic polymer prepared from a monomer of formula (1) as described previously, forming and dewatering the suspension on a wire. In a preferred aspect of the invention, the process further comprises forming and dewatering the suspension on a wire to obtain a wet web containing cellulosic fibres, or paper, and white water, recirculating the white water and optionally introducing fresh water to form a suspension containing cellulosic fibres, and optional fillers, to be dewatered, wherein the amount of fresh water introduced is less than 30 tons per ton of dry paper produced.

The process of this invention results in improved drainage and/or retention when using stocks having high contents of salt, and thus having high conductivity levels, and colloidal materials. Hereby the present invention makes it possible to increase the speed of the paper machine and to use lower dosages of additives to give a corresponding drainage and/or retention effect, thereby leading to an improved papermaking process and economic benefits. The invention is suitably applied to papermaking processes using wood-containing fibre stocks and so-called dirty or difficult stocks, for example those prepared from certain grades of recycled fibres, and/or processes with extensive white water recirculation and limited fresh water supply and/or processes using fresh water having high salt contents, in particular salts of di- and multivalent cations like calcium.

The polymer of the present invention can be added into the stock to be dewatered in amounts which can vary within wide limits depending on, inter alia, type of stock, salt content, type of salts, filler content, type of filler, point of addition, etc. Usually, the present polymer would be added in an amount of at least 0.001%, often at least 0.005% by weight, based on dry stock substance, whereas the upper limit is usually 3% and suitably 1.5% by weight.

In a preferred embodiment of this invention, the polymer of the present invention is used in conjunction with an additional stock additive. Examples of suitable stock additives of this type include anionic microparticulate materials, e.g. anionic organic particles and anionic inorganic particles, water-soluble anionic vinyl addition polymers, low molecular weight cationic organic polymers, aluminium compounds, and combinations thereof.

Anionic inorganic particles that can be used according to the invention include anionic silica-based particles and clays of the smectite type. It is preferred that the anionic inorganic particles are in the colloidal range of particle size. Anionic silica-based particles, i.e. particles based on SiO2 or silicic acid, including colloidal silica, different types of polysilicic acid, colloidal aluminium-modified silica or aluminium silicates, and mixtures thereof, are preferably used. Anionic silica-based particles are usually supplied in the form of aqueous colloidal dispersions, so-called sols.

Anionic silica-based particles suitably have an average particle size below about 50 nm, preferably below about 20 nm and more preferably in the range of from about 1 to about 10 nm.

The anionic inorganic particles may be selected from polysilicic acid and colloidal aluminium-modified silica or aluminium silicate. In the art, polysilicic acid is also referred to as polymeric silicic acid, polysilicic acid microgel, polysilicate and polysilicate microgel, which are all encompassed by the term polysilicic acid used herein. Aluminium-containing compounds of this type are commonly also referred to as polyaluminosilicate and polyaluminosilicate microgel, which are both encompassed by the terms colloidal aluminium-modified silica and aluminium silicate used herein.

Clays of the smectite type that can be used in the process of the invention are known in the art and include naturally occurring, synthetic and chemically treated materials. Examples of suitable smectite clays include montmorillonite/bentonite, hectorite, beidelite, nontronite and saponite, preferably bentonite and especially such bentonite.

Anionic organic particles that can be used according to the invention include highly cross-linked anionic vinyl addition polymers, suitably copolymers comprising an anionic monomer like acrylic acid, methacrylic acid and sulfonated vinyl addition monomers, usually copolymerized with nonionic monomers like (meth)acrylamide, alkyl (meth)acrylates, etc. Useful anionic organic particles also include anionic condensation polymers, e.g. melamine-sulfonic acid sols. Water-soluble anionic vinyl addition polymers that can be used according to the invention include copolymers comprising an anionic monomer like acrylic acid, methacrylic acid and sulfonated vinyl addition monomers, usually copolymerized with nonionic monomers like acrylamide, alkyl acrylates, etc.

Low molecular weight (hereinafter LMW) cationic organic polymers that can be used according to the invention include those commonly referred to and used as anionic trash catchers (ATC). ATC's are known in the art as neutralizing and/or fixing agents for detrimental anionic substances present in the stock and the use thereof in combination with drainage and/or retention aids often provide further improved drainage and/or retention. The LMW cationic organic polymer can be derived from natural or synthetic sources, and preferably it is an LMW synthetic polymer. Suitable organic polymers of this type include LMW highly charged cationic organic polymers such as polyamines, polyethyleneimines, homo- and copolymers based on diallyldimethyl ammonium chloride, (meth)acrylamides and (meth)acrylates. In relation to the molecular weight of the polymer of the present invention, the molecular weight of the LMW cationic organic polymer is preferably lower; it is suitably at least 2,000 and preferably at least 10,000. The upper limit of the molecular weight is usually about 700,000, suitably about 500,000 and preferably about 200,000.

Aluminum compounds that can be used according to the invention include alum, aluminates, aluminium chloride, aluminium nitrate and polyaluminium compounds, such as polyaluminium chlorides, polyaluminium sulphates, polyaluminium compounds containing both chloride and sulphate ions, polyaluminium silicate-sulphates, and mixtures thereof. The polyaluminium compounds may also contain other anions than chloride ions, for example anions from sulfuric acid, phosphoric acid, organic acids such as citric acid and oxalic acid.

The polymer according to the invention and the stock additives described above can be added to the stock in conventional manner and in any order. When using the present polymer and an anionic microparticulate material, notably anionic inorganic particles, it is preferred to add the polymer to the stock before adding the microparticulate material, even if the opposite order of addition may be used. It is further preferred to add the polymer of the present invention before a shear stage, which can be selected from pumping, mixing, cleaning, etc., and to add the anionic particles after that shear stage. When using an LMW cationic organic polymer or an aluminum compound, such components are preferably introduced into the stock prior to introducing the polymer of the present invention, optionally used in conjunction with an anionic microparticulate material. Alternatively, the LMW cationic organic polymer and the polymer of the present invention can be introduced into stock essentially simultaneously, either separately or in admixture. The LMW cationic organic polymer and the polymer of the present invention are preferably introduced into the stock prior to introducing an anionic microparticulate material.

The polymer of the present invention is usually added in an amount of at least 0.001%, often at least 0.005% by weight, based on dry stock substance, and the upper limit is usually 3% and suitably 1.5% by weight. Similar amounts are suitable for water-soluble anionic vinyl addition polymers, if used. When using an anionic microparticulate material in the process, the total amount added is usually at least 0.001% by weight, often at least 0.005% by weight, based on dry substance of the stock, and the upper limit is usually 1.0% and suitably 0.6% by weight. When using anionic silica-based particles, the total amount added is suitably within the range of from 0.005 to 0.5% by weight, calculated as SiO₂ and based on dry stock substance, preferably within the range of from 0.01 to 0.2% by weight. When using an LMW cationic organic polymer in the process, it can be added in an amount of at least 0.05%, based on dry substance of the stock to be dewatered. Suitably, the amount is in the range of from 0.07 to 0.5%, preferably in the range from 0.1 to 0.35%. When using an aluminium compound in the process, the total amount introduced into the stock to be dewatered depends on the type of aluminium compound used and on other effects desired from it. It is for instance well-known in the art to utilize aluminium compounds as precipitants for rosin-based sizing agents. The total amount added is usually at least 0.05%, calculated as Al2O3 and based on dry stock substance. Suitably the amount is in the range of from 0.5 to 3.0%, preferably in the range from 0.1 to 2.0%.

The invention is particularly useful in the manufacture of paper from stocks having high contents of salts of di- and multivalent cations, and usually the content of di- and multivalent cations is at least 200 ppm, suitably at least 300 ppm and preferably at least 400 ppm. The salts can be derived from the stock preparation stage, i.e. from the materials used to form the stock, e.g. water, cellulosic fibres and fillers, in particular in integrated mills where concentrated aqueous fibre suspension from the pulp mill normally is mixed with water to form a dilute suspension suitable for paper manufacture in the paper mill. The salt may also be derived from various additives introduced into the stock, from the fresh water supplied to the process, etc. Further, the content of salts is usually higher in processes where white water is extensively recirculated, which may lead to considerable accumulation of salts in the water circulating in the process. Accordingly, the invention is further suitably used in papermaking processes where white water is extensively recirculated, i.e. with a high degree of white water closure, for example where from 0 to 30 tons of fresh water are used, per ton of dry paper produced, usually less than 20, suitably less than 15, preferably less than 10 and notably less than 5 tons of fresh water per ton of paper. Recirculation of white water obtained in the process suitably comprises mixing the white water with cellulosic fibres and/or optional fillers to form a suspension to be dewatered; preferably it comprises mixing the white water with a suspension containing cellulosic fibres and optional fillers, before the suspension enters the forming wire for dewatering. The white water can be mixed with the suspension before, between, simultaneous with or after introducing the components of drainage and/or retention aids, if used; and before, simultaneous with or after introducing the polymer of the invention. Fresh water can be introduced in the process at any stage; for example, it can be mixed with cellulosic fibres in order to form a suspension, and it can be mixed with a suspension containing cellulosic fibres to dilute it so as to form the suspension to be dewatered, before, simultaneous with or after mixing the stock with white water and before, between, simultaneous with or after inttroducing the stock additives, if used; and before, simultaneous with or after introducing the polymer of the present invention.

The process of this invention may be used for the production of paper. The term “paper”, as used herein, of course include not only paper and the production thereof, but also other cellulosic fibre-containing sheet or web-like products, such as for example board and paperboard, and the production thereof. The process can be used in the production of paper from different types of suspensions of cellulose-containing fibres and the suspensions should suitably contain at least 25% by weight and preferably at least 50% by weight of such fibres, based on dry substance. The suspension can be based on fibres from chemical pulp such as sulphate, sulphite and organosolv pulps, mechanical pulp such as thermomechanical pulp, chemo-thermomechanical pulp, refiner pulp and groundwood pulp, from both hardwood and softwood, and can also be based on recycled fibres, optionally from de-inked pulps, and mixtures thereof.

The polymers of the present invention may be used as a dewatering aid. The treated suspension may be continuously kept in suspension by agitation, for instance when the flocculated suspension is used as a catalyst bed or is being pumped along a flow line, but preferably the flocculated suspension is subjected to solid-liquid separation. Separation may be by sedimentation but preferably it is by centrifugation or filtration. Preferred processes of solid-liquid separation are centrifugal thickening or dewatering, belt pressing, belt thickening and filter pressing. One preferred process of the invention comprises utilising the resultant aqueous composition for flocculating a suspension of suspended solids, especially sewage sludge.

The polymers may be generally used as part of a process for dewatering the suspension and so the flocculated suspension is normally subjected to dewatering. Pressure filtration may be used. This pressure filtration may be by high pressure filtration, for instance on a filter press at 5 to 15 bar for, typically, ½ to 6 hours or low pressure filtration, for instance on a belt press, generally at a pressure of 0.5 to 3 bar, typically 1 to 15 minutes.

The polymers are used by dosing with or without agitation into the suspension, followed by dewatering of the suspension. Optimum results require accurate dosing and the correct degree of agitation during flocculation. If the dose is too low or too high flocculation is inferior. The optimum dose depends upon the content of the suspension and so variations in it, for instance variations in the metal content of industrial sewage effluent, can greatly affect performance. The flocs are very sensitive to shear and agitation, especially if the dosage is not at an optimum, is likely to redisperse the solids as discrete solids. This is a particular problem when the flocculated solids are to be dewatered under shear, for instance on a centrifuge, because if dosage and other conditions are not optimum the centrate is likely to have a high discrete solids content. The polymer can flocculate or dewater waste in order to permit quick and efficient removal of the water from the waste solids. The polymer may be used in its free base or its salt form and may be added to the waste as a solid or as a concentrate in water. It is usual practice to treat each portion of waste with the polymer. A practical procedure is addition of an appropriate amount of a concentrate of the polymer in water to the waste to be treated followed by mechanical manipulation of the treated waste to remove the solids. Other methods of addition include onstream, direct addition, batch addition and addition with other clarification and purification agents. These methods are known to those familiar with the art.

The optimum amount required for treatment of a particular aqueous system will depend upon the identity of the waste solids present. Those familiar with the art will be able to empirically determine the optimum amount required for tests performed on an aliquot of the actual waste. For example, precipitation of the waste solids from the aliquot using differing amounts of polymer will usually reveal which concentration produces clarified water. After introduction of the polymer, the treated particulate matter and water may be separated by siphoning, filtering, centrifuging or by using other common techniques.

The polymers of the present invention are useful for dewatering or flocculating aqueous suspensions or mixtures of organic and inorganic materials or suspensions made entirely of organic material. Examples of such aqueous suspensions include industrial waste from dairies, canneries, chemical manufacturing waste, distillery waste, fermentation waste, waste from paper manufacturing plants, waste from dyeing plants, sewage suspensions such as any type of sludge derived from a sewage treatment plant including digested sludge, activated sludge, raw or primary sludge or mixtures thereof. In addition to the organic material present, the aqueous suspensions may also contain detergents and polymeric materials which will hinder the precipitation process. Modified methods for treatment in view of these factors are known to those familiar with the art.

The following examples further illustrate the present invention.

### EXAMPLE 1

### Synthesis of Monomer

A mixture of methyl acrylate (700 g) and 4-(2-hydroxyethyl)morpholine (700 g) were pre-dried by azeotropic distillation with a small amount of toluene. After cooling the mixture treatment with titanium tetraisopropoxide (30 g) followed by heating the mixture to reflux generated a vapour mixture of methanol and methyl acrylate which was removed to drive the reaction to completion. Further additions of methyl acrylate and titanium tetraisopropoxide were added to maintain the formation of the product monomer in the mixture. The reaction was considered complete when gc analysis of the mixture showed complete conversion of the starting material alcohol. The product monomer was isolated by reduced pressure distillation which afforded pure (4- morpholinoethyl)acrylate (900 g).

### EXAMPLE 2

### Synthesis of Quaternary Ammonium Monomer

(4- morpholinoethyl)acrylate (200 g) was dissolved in chilled acetone (550 g) and purged with an excess of methyl chloride (60 g). The quaternary ammonium monomer product precipitated over the next few days and was removed by filtration, washed with acetone and dried in a vacuum oven.

### EXAMPLE 3

### Synthesis of Polymer

180 g of monomer solution with a 55:40:5 wt % ratio of acrylamide : 4-morpholino-ethyl acrylate quaternary Methyl Chloride: Adipic acid was prepared to which 300 ppm EDTA was added as sequesterant, the adipic acid purely acting as a buffer. The monomer concentration was set at 55% and had a natural pH of 4.1.

To the monomer thermal initiator and one half of a redox couple was added and dispersed. The monomer was then poured into a reaction flask which contained 300 g of an oil phase (Exxsol D40 “RTM”-hydrocarbon solvent) and 3 g of stabiliser both of which had been degassed for 30 minutes with nitrogen. The monomer is dispersed for 3 minutes at a preset stirrer speed during which time the flask contents are adjusted to 25 C. After the dispersion time the second half of the redox couple is added to the dispersed phase which results in the polymersation of the monomer. The reaction is allowed to exotherm to its'peak temperature after which the contents are then heated and distilled under vacuum at 80-85 C. to remove the water present in the bead polymer. After distillation the flask contents are cooled and the bead polymer recovered, washed in acetone to remove residual solvent & stabiliser; filtered and then dried.

### EXAMPLE 4

### Rheological Evaluation

1% active polymer solutions are prepared in deionised water containing different concentrations of salt (calcium chloride). After two hours tumbling time and at a temperature of 20° C. the shear viscosity by Brookfield RVT Viscometer is determined. The speed is 10 rpm and spindle number 2 is used. Table 1 below show the results, the parentheses values show the % reduction in viscosity from the sample containing no calcium chloride.

TABLE 1

| | Brookfield Viscosity (cP) | | |
|---|---|---|---|
| | 28% Cationic | 39% Cationic | 39% Cationic 4- |
| | dimethylaminoethyl | dimethylamino | morpholino-ethyl |
| CaCl2 Concentration | acrylate quaternary | acrylate quaternary | acrylate quaternary |
| (M) | ammonium salt | ammonium salt | methyl chloride |
| 0 | 2880 | 3380 | 3620 |
| 0.005 | 1740 (−40%) | 2060 (−39%) | 2540 (−30%) |
| 0.01 | 1400 (−51%) | 1520 (−55%) | 1940 (−46%) |

These results show that the viscosity of a polymer of the present invention is less adversely affected by increased electrolyte levels.

### EXAMPLE 5

### Sewage Sludge Dewatering, Free Drainage

200 ml aliquots of digested sludge were flocculated using the polymers described below and using appropriate mixing conditions. These were filtered through a portion of belt cloth and the volume collected after 5 seconds was recorded. Each product was evaluated over a dose range in order to get a performance profile.

Polymer represented by a black diamond:

28% dimethylaminoethyl acrylate quaternary ammonium salt, 72% acrylamide copolymer, cationic value of 1.32 meq/g and specific viscosity of 8.6 dl/g.

Polymer represented by a black square:

39% dimethylaminoethyl acrylate quaternary ammonium salt, 61% acrylamide copolymer, cationic value of 2.00 meq/g and specific viscosity of 8.0 dl/g.

Polymer represented by a black triangle:

39% 4-morpholino-ethyl acrylate quaternary methyl chloride salt, 61% acrylamide copolymer, cationic value of 1.67 meq/g and specific viscosity of 8.6 dl/g.

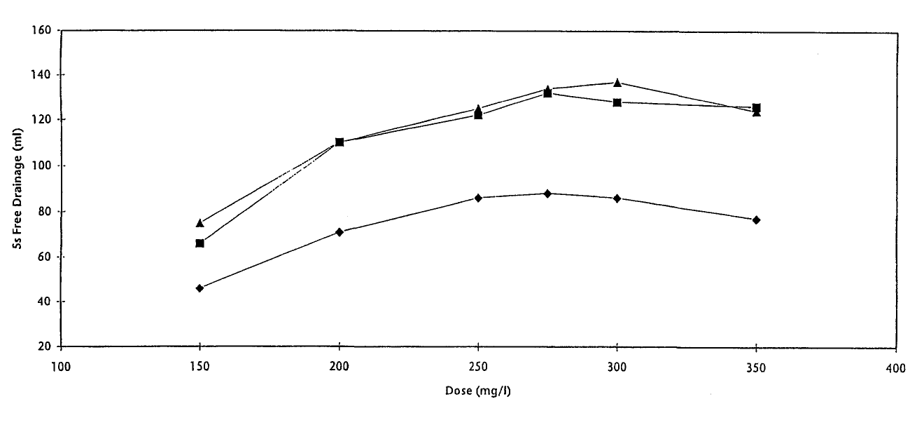

The results are shown in FIG. 1, and clearly show the advantages with respect to free drainage when using polymers of the present invention.

Granular flocs and a relatively clear liquor were also obtained with the instant polymers, compared to gelatinous flocs and a turbid liquor produced by known polymers.

### EXAMPLE 6

### Sewage Sludge Dewatering, Piston Press

200 ml aliquots of digested sludge were flocculated using the same polymers as in example 5 and using appropriate mixing conditions. These were filtered through a portion of belt cloth and allowed to drain for 60 seconds. The thickened substrate was placed in a piston-press in which pressure was applied for 10 minutes. The maximum pressure reached was 100 psi. “Wet” cakes were removed placed in dishes and weighed, placed in the oven (at 110 C.) to dry. Once dried the dishes including cakes were re-weighed and the dry solids was calculated. Each product was evaluated over a dose range in order to get a performance profile.

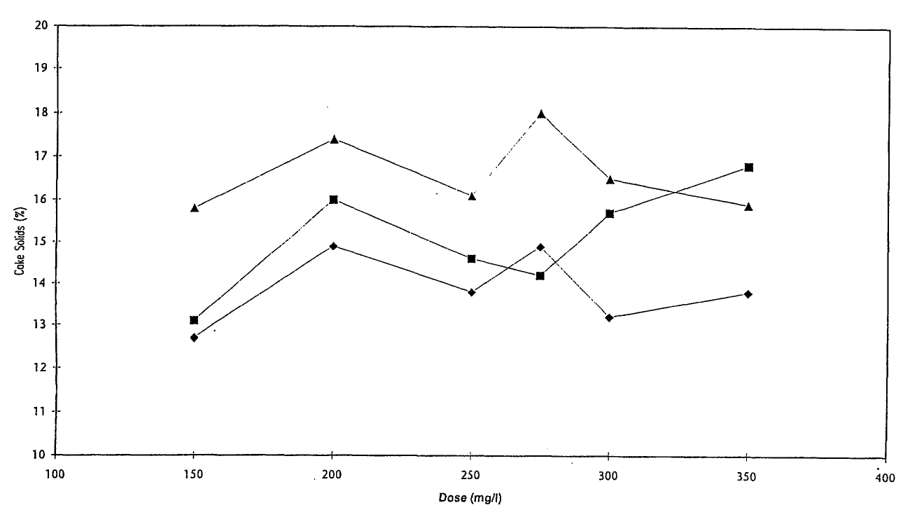

The results are shown in FIG. 2, and clearly show the advantages with respect to cake solids formation when using polymers of the present invention.

Granular flocs and a relatively clear liquor were also obtained with the instant polymers, compared to gelatinous flocs and a turbid liquor produced by known polymers.

### EXAMPLE 7

### Paper Applications, Retention

500 ml aliquots of 1.0% paper stock were flocculated with the same polymers as in example 5, using appropriate mixing conditions. Flocculated samples were added to a Britt Jar with a filter cloth on it's base during agitation and a set volume of filtrate was collected. Known volumes of the filtrate were filtered through pre-weighed filter papers and dried in an oven at 110 C. After drying the filter papers were re-weighed and the First Pass Retention was calculated.

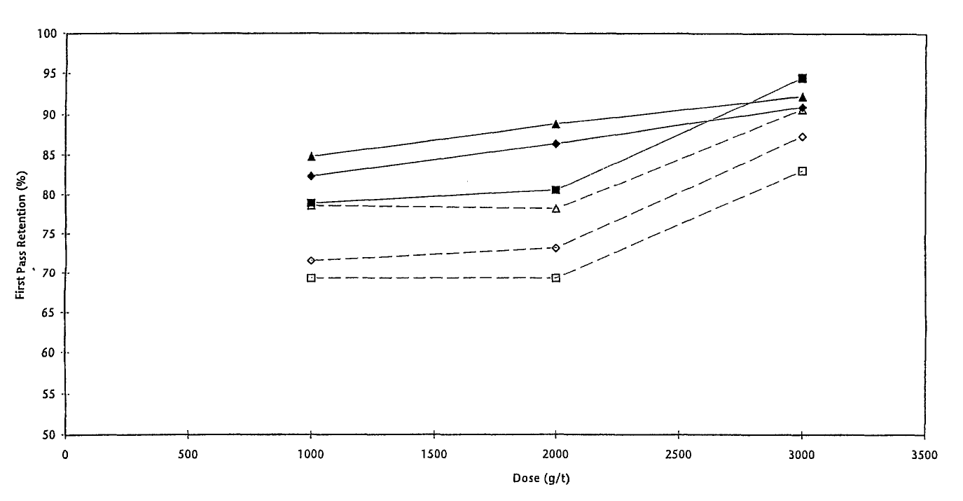

The results are shown in FIG. 3, and clearly show the advantages with respect to increments in first pass retention when using polymers of the present invention.

Electrolyte was added to the paper stock as CaCl₂.6H20 at a concentration of 0.01M and mixed in for fifteen minutes. These results are also shown in FIG. 3, represented by the white square, triangle and diamond. Clearly the polymers of the present invention show less of a reduction in retention when electrolyte is present, when compared to known retention aids.

### EXAMPLE 8

### Paper Applications, Drainage

1000 ml aliquots of 6.5% paper stock were flocculated with the same polymers as in example 5, using appropriate mixing conditions. Flocculated suspensions were added to a drainage apparatus and the time required to collect 500 ml of filtrate was recorded. Each product was evaluated over a dose range in order to get a performance profile.

Electrolyte was added to the paper stock as CaCl₂.6H20 at varying concentrations and mixed in for fifteen minutes.

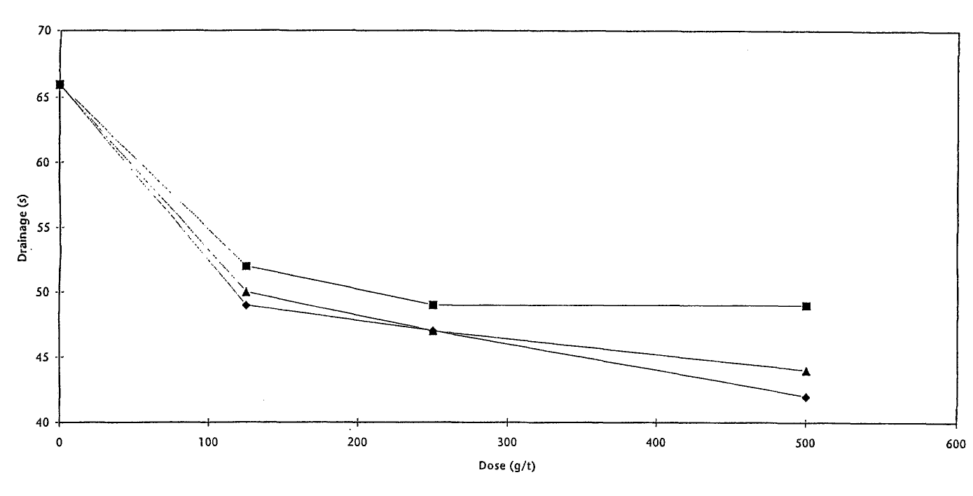

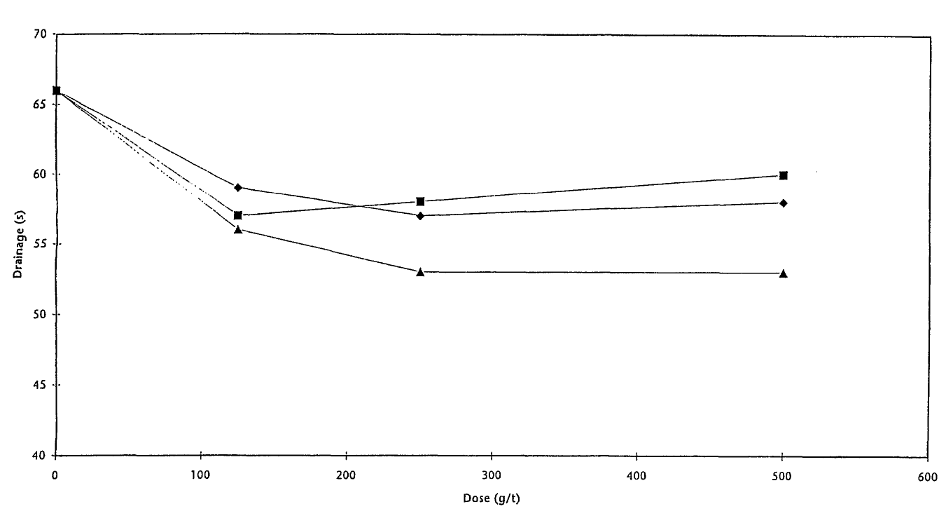

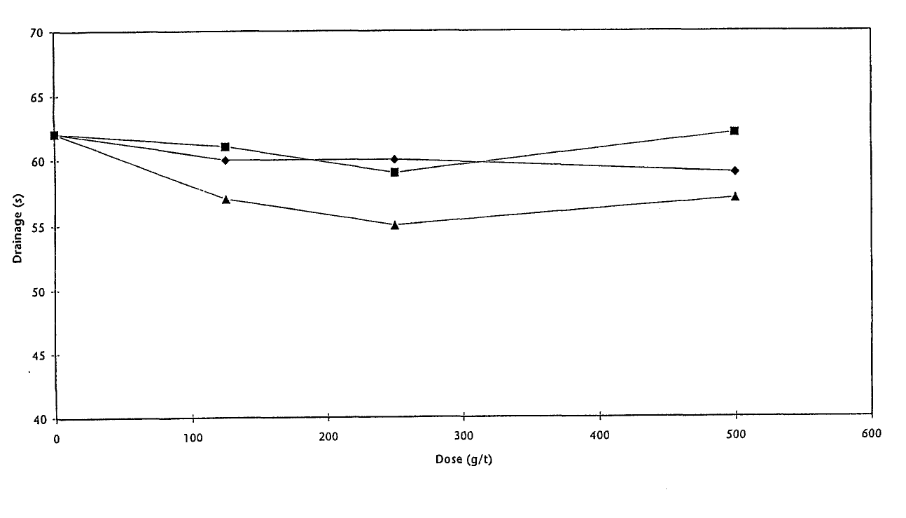

The results are shown in FIGS. 4, 5 and 6, and clearly show the advantages with respect to decreased drainage times when using polymers of the present invention under conditions where electrolyte is present.

### EXAMPLE 9

### Comparative Study

This example is a comparative study of the retention and drainage aid performance of quaternised polymers on paper fine furnish stock at various electrolyte concentrations.

The polymers tested are as follows:

Polymer 1:

40% dimethylaminoethyl acrylate methyl chloride salt, 55% acrylamide copolymer and 5% adipic acid buffer with specific viscosity of 8.0 dl/g.

Polymer 2:

40% 4-morpholino-ethyl acrylate quaternary methyl chloride salt, 55% acrylamide copolymer and 5% adipic acid buffer, with specific viscosity of 8.6 dl/g.

Polymer 3:

47.5% dimethylaminoethyl acrylate benzyl chloride salt, 47.5% acrylamide copolymer and 5% adipic acid buffer, with specific viscosity of 2.2 dl/g.

Polymer 4:

40% dimethylaminoethyl acrylate benzyl chloride salt, 55% acrylamide copolymer and 5% adipic acid buffer, with specific viscosity of 4.8 dl/g.

Paper Applications, Retention

500 ml aliquots of 1.0% paper stock were flocculated with polymers 1 to 4, using appropriate mixing conditions. Flocculated samples were added to a Britt Jar with a filter cloth on it's base during agitation and a set volume of filtrate was collected. Known volumes of the filtrate were filtered through pre-weighed filter papers and dried in an oven at 110 C. After drying the filter papers were re-weighed and the First Pass Retention was calculated.

The results are shown in table 2, and clearly show the advantages with respect to increments in first pass retention when using polymers of the present invention.

TABLE 2

| | | | | |
|---|---|---|---|---|
| Dose (g/t) | Polymer 1 | Polymer 4 | Polymer 3 | Polymer 2 |

First Pass Retention ($)

| | | | | |
|---|---|---|---|---|
| 500 | 89 | 89 | 89 | 92 |
| 1000 | 90 | 93 | 90 | 93 |
| 2000 | 92 | 93 | 92 | 95 |

Electrolyte was added to the paper stock as CaCl₂.6H20 at concentrations of 0.005M and 0.01M and mixed in for fifteen minutes. These results are shown in table 3. Clearly the polymers of the present invention show less of a reduction in retention when electrolyte is present compared to known retention aids.

TABLE 3

| | | | | |
|---|---|---|---|---|
| Dose (g/t) | Polymer 1 | Polymer 4 | Polymer 3 | Polymer 2 |

First Pass Retention (%) at 0.005 M CaCl₂

| | | | | |
|---|---|---|---|---|
| 500 | 86 | 88 | 85 | 90 |
| 1000 | 88 | 91 | 88 | 92 |
| 2000 | 86 | 92 | 89 | 94 |

First Pass Retention (%) at 0.01 M CaCl₂

| | | | | |
|---|---|---|---|---|
| 500 | 87 | 88 | 86 | 89 |
| 1000 | 87 | 91 | 88 | 92 |
| 2000 | 88 | 92 | 90 | 94 |

Paper Applications, Drainage 1000 ml aliquots of 0.5% paper stock were flocculated with the same polymers as in example 5, using appropriate mixing conditions. Flocculated suspensions were added to a drainage apparatus and the time required to collect 500 ml of filtrate was recorded. Each product was evaluated over a dose range in order to get a performance profile.

Electrolyte was added to the paper stock as CaCl₂.6H20 at varying concentrations and mixed in for fifteen minutes.

The results are shown in table 4 and clearly show the advantages with respect to decreased drainage times when using polymers of the present invention, especially under conditions where electrolyte is present.

TABLE 4

| | | | | |
|---|---|---|---|---|
| Dose (g/t) | Polymer 1 | Polymer 4 | Polymer 3 | Polymer 2 |

Drainage (seconds) at 0 M CaCl₂

| | | | | |
|---|---|---|---|---|
| 0 | 69 | 69 | 69 | 69 |
| 250 | 49 | 46 | 48 | 43 |
| 500 | 48 | 41 | 48 | 39 |
| 1000 | 45 | 37 | 45 | 33 |

Drainage (seconds) at 0.005 M CaCl₂

| | | | | |
|---|---|---|---|---|
| 0 | 67 | 67 | 67 | 67 |
| 250 | 59 | 52 | 56 | 51 |
| 500 | 56 | 50 | 56 | 47 |
| 1000 | 55 | 48 | 54 | 45 |

Drainage (seconds) at 0.01 M CaCl₂

| | | | | |
|---|---|---|---|---|
| 0 | 68 | 68 | 68 | 68 |
| 250 | 58 | 53 | 57 | 52 |
| 500 | 58 | 50 | 55 | 48 |
| 1000 | 57 | 47 | 52 | 45 |

1. A compound of the formula (1): 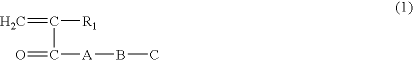
wherein R₁ is H or CH₃, A is O or NH, B is an alkylene group of from 0 to 10 carbon atoms or a hydroxy alkylene group, and C is a cyclic group bonded to A when B is 0, or B by a nitrogen atom which is in the quaternised form.

2. A compound according to claim 1 wherein C is a cyclic group of the formula (2): 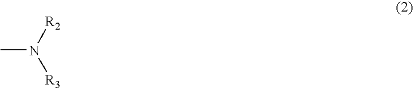
wherein R₂ and R₃ form, together with the adjacent nitrogen atom, a cyclic group which may be saturated or unsaturated, may contain hetero atoms within the cyclic group or as substituents on the cyclic group, and may contain lower alkyl groups.

3. A compound according to claim 1 or 2 wherein C is selected from the group consisting of pyrrolidine, pyrrolidine N-substituted by C₁ to C₄ alkyl, pyrrolidinyl, pyrroline, pyrrolinyl, imidazolidine, imidazolidinyl, imidazoline, imidazolinyl, pirazolidine, pirazolidinyl, pirazoline, pirazolinyl, piperidine, piperidyl, piperazine, piperazine N-substituted by C₁ to C₄ alkyl, piperazinyl, indoline, indolinyl, isoindoline, isoindolinyl, morpholine, morpholinyl, 2H-pyrrole, 2H-pyrrolyl, pyrrole, pyrrolyl, imidazole, imidazolyl, pyrazole, pyrazolyl, pyridine, pyridyl, pyrazine, pyrazinyl, pyrazine, pyrazine para-substituted by C₁ to C₄ alkyl, pyrazinyl, pyrimidine, pyrimidinyl, pyradizine, pyridaznyl, indolizine, indolizinyl, isoindole, isoindolyl, 3H-indole, 3H-indolyl, indole, indolyl, 1H-indazole, indazolyl, purine, purinyl, 4H-quinolizine, 4H-quinolizinyl, isoquinoline, isoquinolyl, quinoline, quinolyl, phthalazine, phthalazinyl, naphthyridine, naphthyridinyl, quinoxaline, quinoxalinyl, quinazoline, quinazolinyl, cinnoline, cinnolinyl, pteridine, pteridinyl, 4aH-carbazole, 4aH-carbazolyl, carbazole, carbazolyl, carboline, carbolinyl, phenanthridine, phenanthridinyl, acridine, acridinyl, perimidine, perimidinyl, phenanthroline, phenanthrolinyl, phenazine, phenazinyl, phenarsazine, phenarsazinyl, phenothiazine, henothiazinyl, furazan, furazanyl, phenoxazine, phenoxazinyl, isothiazole, isoxazole, proline or dehydroproline.

4. A method of preparing a compound of formula (1), 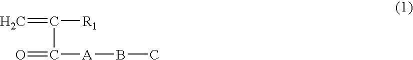
wherein R₁ is H or CH₃, A is O, B is an alkylene group of from 0 to 10 carbon atoms or a hydroxy alkylene group, and C is a cyclic group bonded to A when B is 0, or B by a nitrogen atom which is in the quaternised form, which comprises reacting an ester of the formula (9), 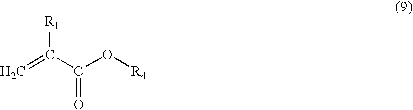
wherein R₄ is a C₁ to C₄ alkyl, with an alcohol of the formula (4).
HO—B—C (4)

5. A cationic or amphoteric organic polymer comprising in polymerised form a monomer containing group C, wherein C is a cyclic group of the formula (2): 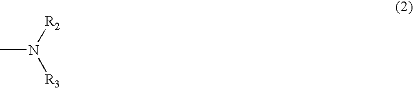
wherein R₂ and R₃ form, together with the adjacent nitrogen atom, a cyclic group which may be saturated or unsaturated, may contain hetero atoms within the cyclic group or as substituents on the cyclic group, and may contain lower alkyl groups.

6. A cationic or amphoteric organic polymer according to claim 5, comprising in polymerised form a monomer of the formula (1) 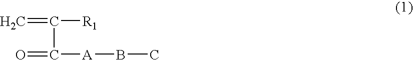
wherein R₁ is H or CH₃, A is O or NH, B is an alkylene group of from 0 to 10 carbon atoms or a hydroxy alkylene group, and C is a cyclic group bonded to A when B is 0, or B by a nitrogen atom which is in the quaternised form, wherein C is a cyclic group of the formula (2): 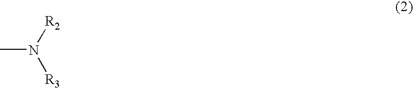
wherein R₂ and R₃ form, together with the adjacent nitrogen atom, a cyclic group which may be saturated or unsaturated, may contain hetero atoms within the cyclic group or as substituents on the cyclic group, and may contain lower alkyl groups.

7. A polymer according to claim 5 or 6 wherein the polymer has an specific viscosity of from 1 to 20 dl/g.

8. A polymer according to any of claims 5 to 7 wherein the polymer is prepared from a monomer mixture comprising from 10 to 100 mole % of a monomer of formula (1) 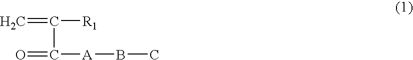
wherein R₁ is H or CH₃, A is O or NH, B is an alkylene group of from 0 to 10 carbon atoms or a hydroxy alkylene group, and C is a cyclic group bonded to A or B by a nitrogen atom, which is in the quaternised form, and from 0 to 90 mole % of other copolymerizable materials.

9. A polymer according to any of claims 5 to 8 wherein the polymer is prepared from a monomer mixture comprising from 20 to 40 mole % of a monomer of formula (1) 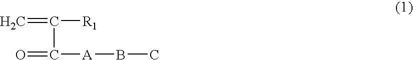
wherein R₁ is H or CH₃, A is O, B is an ethylene group, and C is a morpholine group bonded to B by a nitrogen atom, which is in the quaternised form, and from 80 to 60 mole % of acrylamide.

10. A polymer according to any of claims 5 to 9 wherein the charge density of the polymer is from 1.0 to 4.0 meq/g of dry polymer.

11. A polymer according to any of claims 5 to 10 wherein the polymer is crosslinked.

12. A process of producing paper from a suspension containing cellulosic fibres, which comprises adding to the suspension a polymer according to any of claims 5 to 11.

13. A process of dewatering a sewage sludge suspension which comprises adding to the suspension a polymer according to any of claims 5 to 11.
